(19) United States
(12) Patent Application Publication
(10) Pub. No.: US 2007/0299048 A1
(43) Pub. Date: Dec. 27, 2007

(54) Combination of Gaboxadol and 5Ht2 Antagonists

(76) Inventor: Ruth McKerman, Canterbury (GB)

Correspondence Address: MERCK AND CO., INC, P O BOX 2000, RAHWAY, NJ, 07065-0907, US

(21) Appl. No.: 11/659,565

(22) PCT Filed: Aug. 2, 2005
(86) PCT No.: PCT/GB05/50125

§ 371 Date: Feb. 5, 2007

(30) Foreign Application Priority Data
Aug. 6, 2004 (GB) 0417558.4

(51) Int. Cl. A61K 31/397; A61K 31/437; A61P 25/18; A61P 25/24; A61P 43/00

(52) U.S. Cl. 514/210.21; 514/210.01; 514/302

(57) ABSTRACT

The invention provides the combination of a 5-HT2A antagonist and gaboxadol for use in treatment or prevention of sleep disorders, schizophrenia or depression.

This invention relates to a novel combination of medicaments for therapeutic treatment of humans. In particular, it provides the combination of a 5-HT_{2A} antagonist with gaboxadol for the treatment of sleep disorders, schizophrenia or depression.

Receptors for serotonin (5-hydroxytryptamine, 5-HT) are an important class of G protein-coupled receptors. Serotonin is thought to play a role in processes related to learning and memory, sleep, thermoregulation, mood, motor activity, pain, sexual and aggressive behaviours, appetite, neurodegenerative regulation, and biological rhythms. Not surprisingly, serotonin is linked to pathophysiological conditions such as anxiety, depression, obsessive-compulsive disorders, schizophrenia, suicide, autism, migraine, emesis, alcoholism, and neurodegenerative disorders.

Serotonin receptors are divided into seven subfamilies, referred to as 5-HT₁ through 5-HT₇, inclusive. These subfamilies are further divided into subtypes. For example, the 5-HT₂ subfamily is divided into three receptor subtypes: 5-HT_{2A}, 5-HT_{2B} and 5-HT_{2C}. The human 5-HT_{2C} receptor was first isolated and cloned in 1987, and the human 5-HT_{2A} receptor was first isolated and cloned in 1990. These two receptors are thought to be the site of action of hallucinogenic drugs.

Selective antagonism of the 5-HT_{2A} receptors has been proposed as treatment for a variety of neurological conditions including sleep disorders such as insomnia, psychotic disorders such as schizophrenia, and also depression, anxiety, panic disorder, obsessive-compulsive disorder, pain, eating disorders such as anorexia nervosa, and dependency or acute toxicity associated with narcotic agents such as LSD or MDMA; and for use in controlling the extrapyramidal symptoms associated with the administration of neuroleptic agents (see, for example, WO 00/12090, WO 2004/058722 and US 2003/0130287). They may also be effective in the lowering of intraocular pressure, and in treating menopausal symptoms, in particular hot flushes (see Waldinger et al, Maturitas, 2000, 36, 165-8). Much interest has been shown in the use of 5-HT_{2A} antagonists to treat schizophrenia in the hope of avoiding the extrapyramidal effects associated with typical neuroleptics (which are D2 antagonists) such as haloperidol. However, to date it has not proved possible to elicit a useful anti-psychotic response by antagonism of the 5-HT_{2A} receptors in the absence of simultaneous antagonism of the D2 receptors. There has also been interest in the use of particular 5-HT_{2A} antagonists for treatment of sleep disorders (U.S. Pat. No. 6,613,779 and U.S. Pat. No. 6,277,864).

Gaboxadol (4,5,6,7-tetrahydroisoxazolo[5,4-c]pyridin-3-ol, also known as THIP) is an agonist at the GABA_{A} receptors. As such, it has been proposed for use in treatment of various neurological and psychiatric disorders such as epilepsy, Parkinsonism, schizophrenia and Huntingdon's disease (U.S. Pat. No. 4,278,676) and also for use as an analgesic (U.S. Pat. No. 4,315,934) and as a muscle relaxant (U.S. Pat. No. 4,362,731), but clinically useful therapies in these areas have not, apparently, resulted. More recently, gaboxadol has been proposed for treatment of sleep disorders (WO 97/02813) and diseases relating to reduced neurosteroid activity such as premenstrual syndrome (WO 02/40009). The compound is currently undergoing clinical trials as a treatment for insomnia.

As used herein, the term “gaboxadol” is inclusive of 4,5,6,7-tetrahydroisoxazolo[5,4-c]pyridin-3-ol in free base or zwitterionic form and also of pharmaceutically acceptable acid addition salts thereof such as the hydrochloride salt.

According to the invention, there is provided the combination of a 5-HT_{2A} antagonist and gaboxadol for use in treatment or prevention of sleep disorders, schizophrenia or depression.

Also according to the invention, there is provided a method of treatment or prevention of sleep disorders, schizophrenia or depression comprising administering to a subject in need thereof an effective amount of a 5-HT_{2A} antagonist in combination with an effective amount of gaboxadol.

As used herein, the expression “in combination with” requires that therapeutically effective amounts of both a 5-HT_{2A} antagonist and gaboxadol are administered to the subject, but places no restriction on the manner in which this is achieved. Thus, the two species may be combined in a single dosage form for simultaneous administration to the subject, or may be provided in separate dosage forms for simultaneous or sequential administration to the subject. Sequential administration may be close in time or remote in time, e.g. one species administered in the morning and the other in the evening. The separate species may be administered at the same frequency or at different frequencies, e.g. one species once a day and the other two or more times a day. The separate species may be administered by the same route or by different routes, e.g. one species orally and the other parenterally, although oral administration of both species is preferred, where possible.

According to a further aspect of the invention there is provided a pharmaceutical composition comprising, in a pharmaceutically acceptable carrier, a 5-HT_{2A} antagonist and gaboxadol.

The invention further provides the use, for the manufacture of a medicament for treatment or prevention of sleep disorders, schizophrenia or depression, of a 5-HT_{2A} antagonist and gaboxadol.

The invention further provides a kit comprising a first medicament comprising a 5-HT_{2A} antagonist and a second medicament comprising gaboxadol together with instructions for administering said medicaments sequentially or simultaneously to a patient suffering from a sleep disorder, schizophrenia or depression.

In one embodiment of the invention, the combination of 5-HT_{2A} antagonist and gaboxadol (hereinafter “the Combination”) is for treatment of a sleep disorder, in particular insomnia. This includes primary insomnia, insomnia related to a mental disorder, substance-induced insomnia and circadian rhythm insomnia. “Primary insomnia” as used herein refers to difficulty in falling asleep (increased sleep latency), in maintaining sleep or in experiencing restorative sleep, said difficulty not being caused by a mental disorder or by the physiological effects of taking or withdrawing from specific substances. “Insomnia related to a mental disorder” as used herein refers to difficulty in falling asleep, in maintaining sleep or in experiencing restorative sleep which is caused by a mental disorder such as depression, anxiety or schizophrenia “Substance-induced insomnia” as used herein refers to difficulty in falling asleep, in maintaining sleep or in experiencing restorative sleep which is caused by taking or withdrawing from substances such as caffeine, alcohol, amphetamines, opioids, sedatives, hypnotics and anxiolytics. “Circadian rhythm insomnia” refers to insomnia caused by disruption of an accustomed sleep-wake cycle, e.g. by shift-work or jet-lag.

Sleep comprises two physiological states: Non rapid eye movement (NREM) and rapid eye movement (REM) sleep. NREM sleep consists of four stages, each of which is characterized by progressively slower brain wave patterns, with the slower patterns indicating deeper sleep. So-called delta sleep, stages 3 and 4 of NREM sleep, is the deepest and most refreshing type of sleep. Many patients with sleep disorders are unable to adequately achieve the restorative sleep of stages 3 and 4. In clinical terms, patients' sleep patterns are described as fragmented, meaning the patient spends a lot of time alternating between stages 1 and 2 (semi-wakefulness) and being awake and very little time in deep sleep. The Combination is effective in consolidating sleep patterns so that the patient with previously fragmented sleep can now achieve restorative, delta-wave sleep for longer, more consistent periods of time.

As sleep moves from stage 1 into later stages, heart rate and blood pressure drop, metabolic rate and glucose consumption fall, and muscles relax. NREM sleep makes up about 75% of total sleep time; stage 1 accounting for 5-10% of total sleep time, stage 2 for about 45-50%, stage 3 approximately 12%, and stage 4 13-15%. About 90 minutes after sleep onset, NREM sleep gives way to the first REM sleep episode of the night. REM makes up approximately 25% of total sleep time. In contrast to NREM sleep, REM sleep is characterized by high pulse, respiration, and blood pressure, as well as other physiological patterns similar to those seen in the active waking stage. Hence, REM sleep is also known as “paradoxical sleep”. Sleep onset usually occurs during NREM sleep and takes 10-20 minutes in healthy young adults. The four stages of NREM sleep together with a REM phase form one complete sleep cycle that is repeated throughout the duration of sleep, usually four or five times. The cyclical nature of sleep is regular and reliable; a REM period occurs about every 90 minutes during the night. However, the first REM period tends to be the shortest, often lasting less than 10 minutes, whereas the later REM periods may last up to 40 minutes. With aging, the time between retiring and sleep onset increases and the total amount of night-time sleep decreases because of changes in sleep architecture that impair sleep maintenance as well as sleep quality. Both NREM (particularly stages 3 and 4) and REM sleep are reduced. However, stage 1 NREM sleep, which is the lightest sleep, increases with age.

There are a number of ways to determine whether the onset, duration or quality of sleep (e.g. non-restorative or restorative sleep) is impaired or improved. One method is a subjective determination of the patient, e.g. do they feel drowsy or rested upon waking. Other methods involve the observation of the patient by another during sleep, e.g. how long it takes the patient to fall asleep, how many times does the patient wake up during the night, how restless is the patient during sleep, etc. Another method is to objectively measure the stages of sleep.

Polysomnography is the monitoring of multiple electrophysiological parameters during sleep and generally includes measurement of EEG activity, electroculographic activity and electromyographic activity, as well as other measurements. These results, along with observations, can measure not only sleep latency (the amount of time required to fall asleep), but also sleep continuity (overall balance of sleep and wakefulness) and sleep consolidation (percent of sleeping time spent in delta-wave or restorative sleep) which may be an indication of the quality of sleep.

Human sleep also varies characteristically across the life span. After relative stability with large amounts of slow-wave sleep in childhood and early adolescence, sleep continuity and depth deteriorate across the adult age range. This deterioration is reflected by increased wakefulness and stage 1 sleep and decreased stages 3 and 4 sleep.

The Combination is effective in the treatment of Sleep Disorders by promoting one or more of the following: reduction in sleep onset latency period (measure of sleep induction), reduction in number of night time awakenings, and prolongation of time spent in delta-wave sleep (measure of sleep quality enhancement) without affecting REM sleep.

Therefore, the invention further provides a method of promoting one or more of:
(a) reduction in sleep onset latency period;
(b) reduction in number of night time awakenings; and
(c) prolongation of time spent in delta-wave sleep;
without affecting REM sleep;
   comprising administering to a subject in need thereof an effective amount of a 5-HT_{2A} antagonist in combination with an effective amount of gaboxadol.

It is believed that the combination of medicaments, acting through separate mechanisms, provides a synergistic effect, enabling use of sub-therapeutic doses of the separate components in favourable cases.

In a second embodiment, the Combination is for treatment of schizophrenia. As used herein, “schizophrenia” refers to a psychopathic disorder of unknown origin, which usually appears for the first time in early adulthood and is marked by a number of characteristics, psychotic symptoms, progression, phasic development and deterioration in social behaviour and professional capability in the region below the highest level ever attained. Characteristic psychotic symptoms are disorders of thought content (multiple, fragmentary, incoherent, implausible or simply delusional contents or ideas of persecution) and of mentality (loss of association, flight of imagination, incoherence up to incomprehensibility), as well as disorders of perceptibility (hallucinations), of emotions (superficial or inadequate emotions), of self-perception, of intentions and impulses, of interhuman relationships, and finally psychomotoric disorders (such as catatonia). Other symptoms are also associated with this disorder. (See, American Statistical and Diagnostic Handbook). Use of the Combination enables treatment of schizophrenia without the side effects (in particular extrapyramidal side effects) seen with typical neuroleptics such as haloperidol, and it is believed that the combination of medicaments, acting through separate mechanisms, provides a synergistic effect, enabling use of sub-therapeutic doses of the separate components in favourable cases.

In a third embodiment, the Combination is for treatment of depression.

5-HT_{2A} antagonists suitable for use in the invention may be identified by published methods (see for example Fletcher et al, J. Med. Chem., 2002, 45, 492-503). Preferred 5-HT_{2A} antagonists for use in the invention are selective for the human 5-HT_{2A} receptor over other receptors, notably the human 5-HT_{2C} and other serotonin receptors, dopamine receptors, and IKr (voltage-dependent potassium channel). However, antagonism of the D2 receptor (in addition to the 5-HT_{2A} receptor) may be beneficial when the Combination is used for treatment of schizophrenia.

Suitable 5-HT_{2A} antagonists are disclosed in WO 99/11619, WO 99/11641, WO 99/47511, WO 00/04017, WO 00/05229, WO 00/43362, WO 01/74794, WO 2004/058722, WO 00/107435, WO 01/51469, WO 00/77010 and U.S. Pat. No. 5,169,096. Specific examples of suitable 5-HT_{2A} antagonists include M-100907 (Aventis, WO 00/12090), ACP-103 (CAS RN 359878-17-4, Acadia Pharmaceuticals Inc.), and EMR-62218 (Merck KGaA).

Further examples of suitable 5-HT_{2A} antagonists include the compounds of formula I: 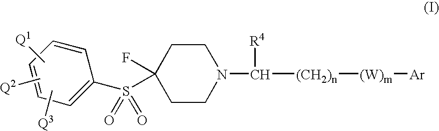
or the pharmaceutically acceptable salts thereof wherein:

Ar is phenyl, benzisothiazol-3-yl or benzthiophen-3-yl, each of which bears substituent groups R¹, R² and R³;

R¹ is hydrogen, fluorine, chlorine, bromine, C₁₋₆ alkyl, C₃₋₆ cycloalkyl, C₂₋₆ alkenyl, C₂₋₆ alkynyl, C₁₋₆ alkoxy, C₂₋₆ alkenyloxy, C₂₋₆ alkynyloxy, or C₁₋₆ alkyl substituted by up to 5-fluorine atoms;

R² is hydrogen, fluorine, chlorine, C₁₋₄ alkyl, C₁₋₄ alkoxy, C₁₋₄ alkyl substituted by up to 5 fluorine atoms or C₁₋₄ alkoxy substituted by up to 5 fluorine atoms;

R³ is hydrogen, fluorine, chlorine, methyl, methoxy, trifluoromethyl, difluoromethyl, trifluoromethoxy or difluoromethoxy;

Q¹ is hydrogen; fluorine; chlorine; bromine; C₁₋₆ alkyl; C₃₋₆ cycloalkyl; C₂₋₆ alkenyl; C₂₋₆ alkynyl; C₁₋₆ alkoxy; C₂₋₆ alkenyloxy; C₂₋₆ alkynyloxy; C₁₋₆ alkyl substituted by up to 5-fluorine atoms; nitrile; COQ⁴ or COQ⁴ where Q⁴ is hydrogen or C₁₋₆ alkyl; NQ⁵Q⁶, CONQ⁵Q⁶ or SO₂NQ⁵Q⁶ where Q⁵ is hydrogen or C₁₋₆ alkyl and Q⁶ is hydrogen or C₁₋₆ alkyl or Q⁵ and Q⁶ are joined to form either a 4-7 membered heterocyclic ring which may also contain one oxygen or one further nitrogen ring atom, which heterocyclic ring may optionally be substituted by up to 3 fluorine atoms or by CF₃, methyl, ethyl or hydroxyl; hydroxyl; nitro; SOQ⁷ or SO₂Q⁷ where Q⁷ is C₁₋₄ alkyl; NQ⁸COQ⁹, NQ⁸CO₂Q⁹ or NQ⁸SO₂Q⁹ where Q⁸ is hydrogen or C₁₋₄alkyl and Q⁹ is hydrogen or C₁₋₄alkyl or is joined to Q⁸ to form a 5-7 membered ring; a heteroaromatic ring of 5 ring atoms 1, 2, 3 or 4 of which may be nitrogen atoms or 1 or 2 of which are nitrogen atoms and 1 of which is an oxygen or sulfur atom or 1 of which is an oxygen or sulfur atom, which heteroaromatic ring optionally being substituted by methyl, ethyl or hydroxyl; or a heteroaromatic ring of 6 ring atoms containing 1 or 2 nitrogen ring atoms or a phenyl group either of which is optionally substituted by 1 or 2 fluorine or chlorine atoms or C₁₋₄alkyl, C₁₋₄alkoxy or trifluoromethyl groups;

Q² is hydrogen, fluorine, chlorine, nitrile, hydroxy, C₁₋₄ alkyl, C₁₋₄ alkoxy, C₁₋₄ alkyl substituted by up to 5 fluorine atoms, or C₁₋₄ alkoxy substituted by up to 5 fluorine atoms;

Q³ is hydrogen, fluorine, chlorine, methyl, methoxy, trifluoromethyl, difluoromethyl, trifluoromethoxy or difluoromethoxy;

or Q² and Q³ are joined to form the residue of a 5, 6 or 7 membered carbocyclic ring;

R⁴ is H or C₁₋₄ alkyl,

m is 0 or 1;

n is 0, 1 or 2; and

W is CH₂, CHF, CH(OH) or CO.

Such compounds are described in International Application WO 2004/101518.

Further examples of suitable 5-HT_{2A} antagonists include the compounds of formula II: 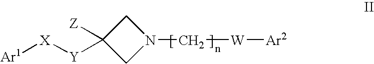
wherein n is 0, 1, 2 or 3;

W is CH₂, CO, CHF or CH(OH);

X is SO₂, CO or CH(OH);

Y is CH₂, CHF or CF₂;

Z is H, F or OH;

Ar¹ is phenyl optionally bearing up to 3 substituents selected from halogen, CN, NO₂, R¹, OR², COR², CO₂R², OCOR¹, SR², S(O)ₜR¹ where t is 1 or 2, N(R²)₂, CON(R²)₂, NR²COR¹ and SO₂N(R²)₂;

Ar² is phenyl or heteroaryl, said heteroaryl having 5 or 6 ring atoms of which one, two or three are selected from N, O and S but not more than one of which is O or S, said heteroaryl optionally being benzo-fused, and said phenyl or heteroaryl optionally bearing up to 3 substituents selected from halogen, CN, NO₂, R¹ and OR²;

R¹ is a hydrocarbon group comprising up to 6 carbon atoms optionally bearing up to 5 fluorine substituents; and

R² is R¹ or H; or two R² groups attached to the same nitrogen atom may complete a morpholine or thiomorpholine ring, or a 5- or 6-membered heterocycle wherein the remaining ring atoms are selected from C and N to a maximum of 4 ring nitrogens in total;

and the pharmaceutically acceptable salts thereof.

Specific Examples of Compounds in Accordance with Formula II Include:

1-[2-(2,4-difluorophenyl)ethyl]-3-{[(4-fluorophenyl)sulfonyl]methyl}azetidine;
1-(4-fluorophenyl)-2-(3-{[(4-fluorophenyl)sulfonyl]methyl}azetidin-1-yl)ethanone;
1-(4-fluorophenyl)-4-(3-{[(4-fluorophenyl)sulfonyl]methyl}azetidin-1-yl)butan-1-one hydrochloride;
2-(3-{[(2,4-difluorophenyl)sulfonyl]methyl}azetidin-1-yl)-1-(4-fluorophenyl)ethanone hydrochloride;
2-(3-fluoro-3-{[(4-fluorophenyl)sulfonyl]methyl}azetidin-1-yl)-1-(4-fluorophenyl)ethanone hydrochloride;
1-[2-(2,4-difluorophenyl)ethyl]-3-{fluoro[(4-fluorophenyl)sulfonyl]methyl}azetidine hydrochloride;
3-{difluoro[(4-fluorophenyl)sulfonyl]methyl}-1-[2-(2,4-difluorophenyl)ethyl]azetidine hydrochloride;
6-fluoro-3-[(3-{[(4-fluorophenyl)sulfonyl]methyl}azetidin-1-yl)methyl]-1,2-benzisothiazole hydrochloride;
6-fluoro-3-[(3-{[(4-fluorophenyl)sulfonyl]methyl}azetidin-1-yl)methyl]-1,2-benzisoxazole hydrochloride; and
1-(4-fluoro-2-methylphenyl)-2-(3-{[(4-fluorophenyl)sulfonyl]methyl}azetidin-1-yl)ethanone.

The compounds of formula II may be obtained by methods disclosed in WO 2005/047246.

Depending on whether they are to be administered together or separately, the 5-HT_{2A} antagonist and gaboxadol are typically supplied as single or multiple pharmaceutical compositions comprising the active species and a pharmaceutically acceptable carrier. Preferably these compositions are in unit dosage forms such as tablets, pills, capsules, powders, granules, sterile parenteral solutions or suspensions, metered aerosol or liquid sprays, drops, ampules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbital, talc, stearic acid, magnesium stearate and dicalcium phosphate, or gum, dispersing agents, suspending agents or surfactants such as sorbitan monooleate and poly(ethylene glycol), and other pharmaceutical diluents, e.g. water, to form a homogeneous preformulation composition containing one or both active species, or pharmaceutically acceptable salts thereof. When referring to these preformulation compositions as homogeneous, it is meant that the active species is or are dispersed evenly throughout the composition so that the composition may be readily subdivided into equally effective unit dosage forms such as tablets, pills and capsules. This preformulation composition is then subdivided into unit dosage forms of the type described above, generally containing from 0.01 to about 500 mg of the active species. Typical unit dosage forms contain from 0.05 to 100 mg, for example 0.05, 0.1, 0.5, 1, 2, 5, 10, 25, 50 or 100 mg, of the active species. Tablets or pills of the pharmaceutical composition(s) can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the pharmaceutical compositions useful in the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acadia, alginate, dextran, sodium carobxymethylcellulose, methylcellulose, poly(ethylene glycol), poly(vinylpyrrolidone) and gelatin.

Suitable dosage formulations and techniques for manufacturing tablets containing gaboxadol and acid addition salts thereof are disclosed in WO 01/22941 and WO 02/094225. Very suitably, gaboxadol is formulated in tablets as a crystalline monohydrate of the zwiterionic form.

For the treatment of sleep disorder, schizophrenia or depression, the relevant 5-HT_{2A} antagonist is administered in combination with gaboxadol at a dose known or estimated to provide occupancy of the 5-HT_{2A} receptors in the brain. Such dosage levels may be determined by standard methods known to those skilled in the art, and will depend, inter alia, on factors such as the bioavailability of the compound in question, its ability to cross the blood-brain barrier, and other pharmacokinetic and pharmacodynamic parameters. Receptor occupancy can be measured in experimental subjects using positron emission techniques and radio-labelled compounds. The frequency of dosing of the relevant compound (e.g. once, twice, three times or four times per day) may be selected according to the phanmacokinetic profile of the compound concerned.

In the case of gaboxadol, suitable dosage is in the range 0.05 to 1.0 mg/Kg per day, typically 0.1 to 0.5 mg/Kg per day. In a preferred embodiment of the invention, gaboxadol or a pharmaceutically acceptable salt thereof is administered as a once a day oral dose equivalent to 15 mg or 20 mg of gaboxadol itself.

In a particular aspect, the invention provides a pharmaceutical formulation comprising, in a pharmaceutically acceptable carrier, a compound of formula II or a pharmaceutically acceptable salt thereof and gaboxadol. Preferably, gaboxadol is present as a crystalline monohydrate of the zwitterionic form. Preferably, said formulation is in the form of a unit dose suitable for oral administration such as a tablet or capsule. In a particular embodiment, each unit dose contains the equivalent of from 10 to 20 mg of gaboxadol free base.

1-9. (canceled)

10. A method for the treatment or prevention of a sleep disorder, schizophrenia or depression comprising administering to a patient in need thereof an effective amount of a 5-HT_{2A} antagonist or a pharmaceutically acceptable salt thereof in combination with an effective amount of gaboxadol or a pharmaceutically acceptable salt thereof.

11. A method for promoting one or more of:
(a) reduction in sleep onset latency period;
(b) reduction in number of night time awakenings; and
(c) prolongation of time spent in delta-wave sleep;
without affecting REM sleep, in a patient in need thereof;
comprising administering to the patient an effective amount of a 5-HT_{2A} antagonist or a pharmaceutically acceptable salt thereof in combination with an effective amount of gaboxadol or a pharmaceutically acceptable salt thereof.

12. A pharmaceutical composition comprising, in a pharmaceutically acceptable carrier, a 5-HT_{2A} antagonist or a pharmaceutically acceptable salt thereof and gaboxadol or a pharmaceutically acceptable salt thereof.

13. The method of claim 10 wherein gaboxadol is in the form of a crystalline monohydrate of its zwitterionic form.

14. The method of claim 10 wherein the 5-HT_{2A} antagonist is selective for the human 5-HT_{2A} receptor over one or more of the human 5-HT_{2C} receptor, dopamine receptors and IKr.

15. The method of claim 10 wherein the 5-HT_{2A} antagonist is selected from a compound of the formula II: 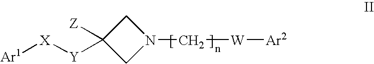
wherein n is 0, 1, 2 or 3;
W is CH₂, CO, CHF or CH(OH);
X is SO₂, CO or CH(OH);
Y is CH₂, CHF or CF₂;
Z is H, F or OH;
Ar¹ is phenyl optionally bearing up to 3 substituents selected from halogen, CN, NO₂, R¹, OR², COR², CO₂R², OCOR¹, SR², S(O)ₜR¹ where t is 1 or 2, N(R²)₂, CON(R²)₂, NR²COR¹ and SO₂N(R²)₂;
Ar² is phenyl or heteroaryl, said heteroaryl having 5 or 6 ring atoms of which one, two or three are selected from N, O and S but not more than one of which is O or S, said heteroaryl optionally being benzo-fused, and said phenyl or heteroaryl optionally bearing up to 3 substituents selected from halogen, CN, NO₂, R¹ and OR²;
R¹ is a hydrocarbon group comprising up to 6 carbon atoms optionally bearing up to 5 fluorine substituents; and
R² is R¹ or H; or two R² groups attached to the same nitrogen atom may complete a morpholine or thiomorpholine ring, or a 5- or 6-membered heterocycle wherein the remaining ring atoms are selected from C and N to a maximum of 4 ring nitrogens in total;
or a pharmaceutically acceptable salt thereof.

16. The method of claim 15 wherein the 5-HT_{2A} antagonist is selected from:
1-[2-(2,4-difluorophenyl)ethyl]-3-{[(4-fluorophenyl)sulfonyl]methyl}azetidine;
1-(4-fluorophenyl)-2-(3-{[(4-fluorophenyl)sulfonyl]methyl}azetidin-1-yl)ethanone;
1-(4-fluorophenyl)-4-(3-{[(4-fluorophenyl)sulfonyl]methyl}azetidin-1-yl)butan-1-one hydrochloride;
2-(3-{[(2,4-difluorophenyl)sulfonyl]methyl}azetidin-1-yl)-1-(4-fluorophenyl)ethanone hydrochloride;
2-(3-fluoro-3-{[(4-fluorophenyl)sulfonyl]methyl}azetidin-1-yl)-1-(4-fluorophenyl)ethanone hydrochloride;
1-[2-(2,4-difluorophenyl)ethyl]-3-{fluoro[(4-fluorophenyl)sulfonyl]methyl}azetidine hydrochloride;
3-{difluoro[(4-fluorophenyl)sulfonyl]methyl}-1-[2-(2,4-difluorophenyl)ethyl]azetidine hydrochloride;
6-fluoro-3-[(3-{[(4-fluorophenyl)sulfonyl]methyl}azetidin-1-yl)methyl]-1,2-benzisothiazole hydrochloride;
6-fluoro-3-[(3-{[(4-fluorophenyl)sulfonyl]methyl}azetidin-1-yl)methyl]-1,2-benzisoxazole hydrochloride; and
1-(4-fluoro-2-methylphenyl)-2-(3-{[(4-fluorophenyl)sulfonyl]methyl}azetidin-1-yl)ethanone;
or a pharmaceutically acceptable salt thereof.
